(19) United States
(12) Patent Application Publication
(10) Pub. No.: US 2025/0043267 A1
(43) Pub. Date: Feb. 6, 2025

(54) ENGINEERED BACTERIAL TYROSYL-TRNA SYNTHETASE MUTANTS FOR INCORPORATING UNNATURAL AMINO ACIDS INTO PROTEINS

(71) Applicant: TRUSTEES OF BOSTON COLLEGE, Chestnut Hill, MA (US)

(72) Inventors: Abhishek Chatterjee, Lexington, MA (US); Katherine T. Grasso, Chandler, AZ (US); Soumya Jyoti Singha Roy, San Diego, CA (US)

(21) Appl. No.: 18/705,012

(22) PCT Filed: Oct. 28, 2022
(86) PCT No.: PCT/US2022/078860

§ 371 Date: Apr. 26, 2024

Related U.S. Application Data
(60) Provisional application No. 63/263,268, filed on Oct. 29, 2021.

(51) Int. Cl. C12N 9/00; C12P 21/00

(52) U.S. Cl. CPC C12N 9/93; C12P 21/00; C12Y 601/01001

(57) ABSTRACT

Compositions of bacterial tyrosyl-tRNA synthetase mutants that enable site-specific incorporation of numerous unnatural amino acids into proteins expressed in eukaryotic cells or in engineered ATM E. coli and methods of use are described. Compositions of novel unnatural amino acids that can be incorporated using these mutant bacterial tyrosyl-tRNA synthetase are also described.

### RELATED APPLICATIONS

This application claims the benefit under 35 U.S.C. § 119 (e) of U.S. Provisional Application No. 63/263,268, filed on Oct. 29, 2021, which is incorporated herein by reference in its entirety.

### GOVERNMENT SUPPORT

This invention was made with government support under grant number R35GM136437, awarded by the National Institutes of Health. The government has certain rights in this invention.

### REFERENCE TO AN ELECTRONIC SEQUENCE LISTING

The contents of the electronic sequence listing (03420012WO1_ST26.xml; Size: 10,485 bytes; and Date of Creation: Oct. 24, 2022) is herein incorporated by reference in its entirety.

### FIELD OF THE INVENTION

The present disclosure is directed to the field of biotechnology describing compositions of an engineered tyrosyl-tRNA synthetase used for the site-specific incorporation of a variety of unnatural amino acids into recombinant proteins expressed in live cells

### BACKGROUND OF THE INVENTION

The site-specific incorporation of unnatural amino acids (UAAs) through genetic code expansion (GCE) enables the study and manipulation of protein structure/function by introducing novel chemical modalities (i.e., photoaffinity probes, bioconjugation handles, post-translational modifications). To execute GCE, a non-cross reactive (orthogonal) aminoacyl-tRNA synthetase (aaRS), an orthogonal tRNA, and a blank codon are all needed. Traditionally, an orthogonal aaRS is imported into a host organism from an evolutionarily distant domain of life. Then, the substrate specificity of the orthogonal aaRS is altered using directed evolution. First, rationally selected key amino acid residues in the active site of the orthogonal aaRS are randomized to create a library of mutants. Second, from this pool of mutants, members active for the selective incorporation of the desired UAA are enriched through a double sieve selection scheme followed by their characterization.

Evolution of each new mutant UAA-tRNA synthetase (UAA-RS) is not trivial. This is due to the technical complexities of building an orthogonal aaRS library that is large enough to potentially contain a mutant solution. Some evolved UAA-RS variants are polyspecific (able to incorporate more than one UAA that are structurally similar). Polyspecific UAA-RS mutants are advantageous because they circumvent the limitations of having to perform time-consuming and laborious directed evolution experiments targeted to each individual UAAs. Consequently, the identification of new polyspecific UAA-RSs significantly broadens the toolbox of incorporable UAAs, enabling innovative applications and manipulations of protein structure.

### SUMMARY OF THE INVENTION

A bacteria-derived tyrosyl-tRNA synthetase (TyrRS)/tRNA pair has been previously engineered to site-specifically incorporate a small set of UAAs into proteins expressed in eukaryotic cells such as yeast and mammalian cells. (See for example WO2020/219708, the teachings of which are incorporated herein in their entirety.) Recently, altered translational machinery (ATM) E. coli strains have been developed, where the endogenous TyrRS/tRNA pair is substituted with an archaeal counterpart. (See for example, U.S. Pat. No. 10,717,975, the teachings of which are incorporated herein in their entirety.) Since this ATM-Tyr (ATMY) E. coli strain lacks the endogenous bacterial TyrRS/tRNA pair, it can be reintroduced therein for incorporating an unnatural amino acid residue. Consequently, developing engineered mutants of the bacterial TyrRS that charges new UAAs would facilitate their incorporation into proteins expressed both in eukaryotic cells as well as any ATMY E. coli.

The present invention encompasses novel engineered mutants/variants of the bacterial aminoacyl-tyrosyl-tRNA synthetase (TyrRS) that selectively charge a series of unnatural amino acids (UAAs or Uaas, also referred to herein a non-canonical amino acids or ncAAs). With the exception of the analog pBPA, the UAAs described herein are novel. Compositions are described herein comprising a variant E. coli tyrosyl-tRNA synthetase (EcTyr-RS) wherein the EcTyr-RS preferentially aminoacylates an E. coli tyrosyl tRNA (Ec-tRNA^{Tyr}) with a tyrosine analog over the naturally-occurring tyrosine amino acid. These Uaas can be site-specifically incorporated into proteins expressed in bacterial or eukaryotic cells using these engineered TyrRS mutants and their cognate tRNA. More specifically, these UAAs are described herein.

In particular, the current invention encompasses a composition comprising a mutant/variant of E. coli tyrosyl-tRNA synthetase (EcTyrRS) wherein the wild-type EcTyr-RS comprises the amino acid sequences as published in the NCBI database for the K-12 E. coli strain (ncbi.nlm.nih.gov/protein/BAE77907), or E. coli strain (ncbi.nlm.nih.gov/protein/.BAA15398.2 K-12 E. coli strain, substrain DH10B (ncbi.nlm.nih.gov/protein and Gen Bank accession number ACB02843) or E. coli strain MG1655 NCBI Reference Sequence NP_416154.1. The variant EcTyr-RS preferentially aminoacylates an E. coli tyrosyltRNA (EctRNA^{tyr}) with a tyrosine analog over the naturally-occurring tyrosine amino acid, wherein the variant EcTyr-RS comprises the amino acid sequence of SEQ ID NO:1, or a homologous EcTyr-RS comprising an amino acid sequence with at least about 80%, 85%, 90%, 95%, 96%, 97%, 98% or 99% sequence identity with the full-length SEQ ID NO: 1 and comprising mutated amino acid positions. The variant EcTyrRS is mutated at critical amino acid residues at active-sites as shown in FIG. 5A. Substitutions of suitable replacement amino acid residues at these sites are also show in FIG. SB (SEQ ID NOS. 2-5, poly TyrRS-9; polyYyrRS-6, polyTyrRS-11 and polyTyrRS-16 respectively).

Specifically, the variant E. coli tyrosyl-tRNA synthetase comprises SEQ ID NO: 1, or an amino acid sequence with at least about 90% sequence identity with the full-length SEQ ID NO:1, with one, or more mutations at the following positions: Y37; L71; N126; D182; F183; L186 or D2651. More specifically, encompassed by the present invention is a variant Ec-tyrosyl-RS wherein the tyrosine (Y) at position 37 is replaced with glycine (G) or cysteine (C), the leucine (L) at position 71 is replaced with valine (V), cysteine (C) or isoleucine (1), the aspartic acid (D) at position 182 is replaced with cysteine (C) or serine(S), the phenylalanine (F) at position 183 is replaced with tyrosine (Y) or methionine ((M), the leucine (L) at position 186 is replaced with cysteine (C), arginine (R) or glycine (G), the aspartic acid (D) at position 265 is replaced with arginine (R) and the asparagine (N) at position 126 is conserved.

Examples of variant amino acid sequences of the EcTyr-RS molecules of the present invention encompassing such amino acid mutations/substitutions/conservations (conserved amino acid residues that are the same as in the wild type EcTyr-RS) are shown in FIG. 5B comprising SEQ ID NO:2 through SEQ ID NO:5. Specifically, as shown in FIG. 5B, poly-TyrRS-9 comprises SEQ ID NO: 1 with the following mutations/substitutions/conservations: Y37G; L71V; N126N; D182C; F183Y; L186C and D265R designated SEQ ID NO:2.

Poly-TyrRS-6 of FIG. 5B comprises SEQ ID NO: 1 with the following mutations/conserved amino acid residues: Y37C; L71C; N126N; D182S; D183M; L186A and D265R and designated SEQ ID NO:3.

Poly-TyrRS-11 of FIG. 5B comprises SEQ ID NO: 1 with the following mutations/substitutions/conservations: Y37G; L71V; N126N; D182S; F183M; L186C; and D265R designated as SEQ ID NO: 4.

Poly-TyrRS-16 of FIG. 5B comprises SEQ ID NO: 1 with the following mutations/substitutions/conservations: Y37C; L711; N126N; D182S; F183M; L186G and D265R designated as SEQ ID NO:5.

Importantly, the Ec-tyrosyl-RS variants of the present invention are polyspecific—that is, the variant Tyr-RS enables site-specific incorporation of numerous unnatural amino acids (e.g., as shown in FIG. 1) in proteins of interest expressed in eukaryotic cells or in the genetically-engineered ATM E. coli as described herein. The mutant polyspecific EcTyr-tRNA-synthetases as described herein can incorporate several UAAs (non-sulfonated tyrosine analogs) into proteins expressed in bacteria and eukaryotes with high fidelity and efficiency (hence the term “polyspecific”). Examples of such tyrosine analogs are selected from the group consisting of: pBPA, pAEY, pAzAcF, pAzPrAmF, alkene amide, pAcrF, pAlkAcF, pCAcF, pFAcF and propargyl carbamate (see FIG. 1). More specifically, the novel UAAs are selected from the group consisting of: pAEY, pAzAcF, pAzPrAmF, alkene amide, pAcrF, pAlkAcF, pCAcF, pFAcF and propargyl carbamate.

The Tyr-tRNA synthetases encompassed by the present invention further include homologous archaeal or bacteria-derived Tyr-RNA synthetases with active-site residues substituted with mutations as described herein. The homologous Tyr-RS comprises an amino acid sequence with at least about 80%, 85%, 90%, 95%, 96%, 97%, 98% or 99% sequence identity with the full-length SEQ ID NO:1. The homologous Tyr-RS can be mutated at its active-site residues corresponding to the mutations as described herein for the E. coli Tyr-RS, for example at positions: Y37; L71, N126; D182; F183; L186 or D2651.

Such homologous TyrRS genes can be identified by techniques known to those of skill in the art, for example by performing sequence identity/homology searches of TyrRS genetic sequence databases to identify TyrRS gene sequences with, for example, about 80% sequence identity; about 85% sequence identity; about 90% sequence identity; about 95% sequence identity or greater than about 95% sequence identity, which are substantially homologous, or highly homologous to the E. coli TyrRS described herein. Such homologous TyrRS genes suitable for use as described herein may contain sequence variation from the E. coli Tyr-RS wherein such sequence variations do not affect the functionality (aminoacyl activity) of the RNA synthetase. Such nucleotide variations can be defined as conservative sequence variations or substitutions.

It is noted that the E. coli tyrosyl-tRNA synthetases and O-sulfotyrosine analogs as described in WO 2020/219708 are not encompassed by the present invention and are specifically excluded.

Also encompassed by the present invention are polynucleotide sequences that encode the variant polyspecific tyrosyl-tRNA synthetases as described herein, and polynucleotide sequences that hybridize to those polynucleotide sequences under highly stringent conditions over substantially the entire length of the nucleotide sequence, as well as the polypeptides encoded by the polynucleotides. For example, the nucleotide sequence SEQ ID NO:6 encodes the variant “polyTyrRS-9” as described herein.

The present invention further encompasses tRNA compositions wherein the tRNA anti-codon loop is modified (e.g., mutated) to specifically bind to (e.g., recognize) an amber (UAG/TAG) codon as described herein. tRNA compositions comprising the ochre codon (TAA/UAA) or the opal codon (UGA/TGA) are also encompassed by the present invention. In particular, the present invention encompasses compositions wherein the tRNA is the E. coli tyrosyl tRNA, or another homologous bacteria-derived tRNA, wherein the polynucleotide sequence comprises, for example, SEQ ID NO:7, or a tyrosyl-tRNA sequence with about 80%; about 85%; about 90%, about 95% or greater than about 95% sequence identity with SEQ ID NO:7, and with an anti-codon loop comprising a sequence that specifically binds to a selector sequence of an mRNA comprising an amber codon. Importantly, the tRNA EcTyr UAG described herein is an amber suppressor suitable for use in both genetically-engineered bacteria and eukaryotes. For E. coli tyrosyl RNA sequences suitable for use in this invention, also see, for example, sequences described in Cell Chem. Biol. 25, 13-4-1312 (2018).

It is important to note that the modified tRNA of E. coli, or a homologous bacteria-derived tRNA, can be combined with an RNA synthetase of another homologous bacteria-derived RNA synthetase to produce novel combinations for unnatural amino acid, e.g., tyrosine analog, incorporation into proteins. Additionally, a combination of two distinct tRNA-RS/tRNA pairs can be combined. For example, the EcTyr-RS/tRNA pair described herein can also be combined with other suitable tRNA-RS/tRNA pairs which respond to other codons such as an opal suppressor, to site-specifically incorporate two distinct unnatural amino acids into polypeptide/proteins expressed in eukaryotic cells.

The present invention also encompasses the following novel UAAs as shown in FIG. 1: pAEY, pAzAcF, pAzPrAmF, alkene amide, pAcrF, pAlkAcF, pCAcF, pFAcF and propargyl carbamate. The variant EcTyr-RS enzymes of the present invention preferentially aminoacylate a corresponding E. coli tyrosyl-tRNA with these novel tyrosine analogs over the naturally-occurring tyrosine amino acid reside.

Also encompassed by the present invention are bacterial or eukaryotic cells comprising the aminoacyl-Tyr-RS variants described herein, and in particular, comprising nucleic acid sequences encoding these Tyr-RS variants. In one embodiment herein is a cell (as used herein the singular term “cell” also encompasses the plural “cells”) comprising a variant E. coli tyrosyl-tRNA synthetase (EcTyr-RS), wherein the variant EcTyr-RS preferentially aminoacylates an E. coli tyrosyl-tRNA with a tyrosyl analog, and an orthogonal E. coli-tyrosyl tRNA (Ec-tRNATy) as a pair, wherein the variant EcTyr-RS comprises the amino acid sequence of SEQ ID NO: 1 or an amino acid sequence with at least about 90% sequence identity with the full-length SEQ ID NO: 1, wherein the EcTyr-RS is mutated relative to SEQ ID NO:1 at amino acid residues tyrosine (Y) 37, leucine (L) 71, aspartic acid (D) 182, phenylalanine (F) 183, leucine (L) 186 and aspartic acid (D) 265 and N126 is conserved.

More specifically, the cell comprises a variant EcTyr-RS comprising the amino acid sequence SEQ ID NO:1, or an amino acid sequence with at least about 90% sequence identity with the full-length SEQ ID NO:1, wherein the tyrosine (Y) at position 37 is replace with glycine (G) or cysteine (C), the leucine (L) at position 71 is replaced with valine (V), cysteine (C) or isoleucine (I), the aspartic acid (D) at position 182 is replaced with cysteine (C) or serine(S), the phenylalanine (F) at position 183 is replaced with tyrosine (Y) or methionine ((M), the leucine (L) at position 186 is replaced with cysteine (C), arginine (R) or glycine (G), the aspartic acid (D) at position 265 is replaced with arginine (R) and the asparagine (N) at position 126 is conserved. For example, the cell as described herein comprises a variant E. coli tyrosyl-tRNA synthetase comprising an amino acid sequence selected from the group consisting of: SEQ ID NO:2; SEQ ID NO:3; SEQ ID NO: 4 or SEQ ID NO:5.

The cells of the present invention can be bacterial or eukaryotic cells. In one embodiment the eukaryotic cell is a mammalian cell. In another embodiment the bacterial cell is E. coli. E. coli cells encompassed herein include any ATMY (altered translational machinery-tyrosine) E. coli and in particular, the ATMY4 strain of E. coli.

Also encompassed is a cell, wherein the cell comprises a polynucleotide encoding the amino acid sequence of the variant E. coli tyrosyl-tRNA synthetase as descried herein. For example, the polynucleotide comprises the sequence of SEQ ID NO:6, or a nucleotide sequence with at least about 90% sequence identity of SEQ ID NO:6.

Cells encompassed by the present invention also include the additional components/nucleic acid or amino acid sequences required for the cells to produce/express the proteins comprising the UAAs described herein. Such components are known to those of skill in the art and can be as described in U.S. Pat. 10,717,975, the teachings of which are incorporated herein in their entirety.

Further encompassed by the present invention are methods of producing proteins incorporating unnatural amino acids in a site-specific manner using a variant Tyr-RS as described herein. The steps of a method of producing a protein or peptide of interest in a cell with one, or more, tyrosyl analogs at specified amino acid residue positions in the protein or peptide, comprises the following: a) culturing the cell comprising a nucleic acid encoding a variant E. coli tyrosyl-RS as described herein in a culture medium under conditions suitable for growth, wherein the cell comprises a nucleic acid encoding a protein or peptide of interest with one, or more, amber selector codons incorporated at the one, or more specified positions in the protein or peptide, wherein the cell further comprises a nucleic acid encoding an Ec-tRNA^{Tyr} that recognizes the selector codon, and b) contacting the cell culture medium with one, or more, tyrosyl analogs under conditions suitable for incorporation of the one, or more, tyrosyl analogs into the protein in response to the selector codon, thereby producing the protein or peptide of interest with one, or more tyrosyl analogs at specified positions in the protein or peptide.

Specifically encompassed herein is a method of site-specifically incorporating one, or more, tyrosyl analogs into a protein or peptide of interest in a cell, the method comprising the steps of: a) culturing the cell in a culture medium under conditions suitable for growth, wherein the cell comprises a nucleic acid encoding a protein or peptide of interest with one, or more, amber selector codons incorporated at one, or more at specific sites in the protein or peptide, and wherein the cell further comprises a nucleic acid encoding a variant E. coli tyrosyl-tRNA synthetase (EcTyr-RS), wherein the EcTry-RS preferentially aminoacylates an E. coli tyrosyl tRNA (Ec-tRNA^{Try}) that recognizes the amber selector codon, wherein the variant EcTyr-RS comprises the amino acid sequence of SEQ ID NO:1, or an amino acid sequence with at least 90% sequence identity with the full-length SEQ ID NO:1, wherein the EcTyr-RS is mutated relative to SEQ ID NO:1 at amino acid residues tyrosine (Y) 37, leucine (L) 71, aspartic acid (D) 182, phenylalanine (F) 183, leucine (L) 186 and aspartic acid (D) 265; and b) contacting the cell culture medium with one, or more, tyrosyl analogs under conditions suitable for incorporation of the one, or more, tyrosyl analogs into the protein or peptide at the sites of the selector codon(s), thereby producing the protein or peptide of interest with one, or more site-specifically incorporated tyrosyl analogs.

In particular, the variant E. coli tyrosyl-tRNA synthetase (EcTyr-RS) comprises SEQ ID NO:1, or an amino acid sequence with at least about 90% sequence identity with the full-length SEQ ID NO:1, wherein the tyrosine (Y) at position 37 is replace with glycine (G) or cysteine (C), the leucine (L) at position 71 is replaced with valine (V), cysteine (C) or isoleucine (1), the aspartic acid (D) at position 182 is replaced with cysteine (C) or serine(S), the phenylalanine (F) at position 183 is replaced with tyrosine (Y) or methionine ((M), the leucine (L) at position 186 is replaced with cysteine (C), arginine (R) or glycine (G), the aspartic acid (D) at position 265 is replaced with arginine (R) and the asparagine (N) at position 126 is conserved.

For example, the variant E. coli tyrosyl-tRNA synthetase can comprise an amino acid sequence selected from the group consisting of: SEQ ID NO:2; SEQ ID NO:3; SEQ ID NO: 4 or SEQ ID NO:5 and the tyrosyl analog is selected from the group consisting of: pBPA, pAEY, pAzAcF, pAzPrAmF, alkene amide, pAcrF, pAlkAcF, pCAcF, pFAcF and propargyl carbamate.

As described herein, the cell can be an E. coli cell or a eukaryotic cell. In one embodiment the eukaryotic cell is a mammalian cell. In another embodiment the E. coli cell is the ATMY strain of E. coli cell (for example as described in U.S. Pat. No. 10,717,975, the teachings of which are incorporated herein in their entirety). In particular, the strain of E. coli cell is ATMY4, as described herein.

Also encompassed by the present invention are methods of incorporating two, or more unnatural amino acids at specified positions in a polypeptide/protein expressed in a cell. In these methods the cell further comprises a second tRNA/RS pair that is orthogonal to the cell, wherein the second pair recognizes a selector codon in the protein but does not cross-react with the first RS/tRNA pair (e.g., EcTyr-RS/tRNA^{tyr}). The method is performed as above (or in a similar manner) wherein the protein expressed/produced contains one, or more tyrosine analogs and one, or more, distinct unnatural amino acid other than a tyrosine analog incorporated by the first RS/tRNA pair.

Further encompassed by the present invention are kits comprising variant Ec-tyrosyl-RS molecules as described herein. Specifically encompassed herein is a kit for producing a protein or peptide of interest in a cell, wherein the protein or peptide comprises one, or more tyrosyl analogs. The components of the kit comprise a container containing a polynucleotide sequence encoding an Ec-tRNA^{Tyr} that recognizes an amber selector codon; and a container containing a polynucleotide encoding a variant E. coli tyrosyl tRNA synthetase that preferentially aminoacylates the Ec-tRNA^{Tyr} with a tryrosyl analog, wherein the EcTry-RS comprises the amino acid sequence of SEQ ID NO:1, or an amino acid sequence with at least about 90% sequence identity with the full-length SEQ ID NO: 1, wherein the EcTyr-RS is mutated relative to SEQ ID NO: 1 at amino acid residues tyrosine (Y) 37, leucine (L) 71, aspartic acid (D) 182, phenylalanine (F) 183, leucine (L) 186 and aspartic acid (D) 265. Also included in the kit are buffers, diluents and other components necessary for producing a protein or peptide of interest with site-specific incorporation of one, or more UAAs as described herein.

In one embodiment, the kit comprises a nucleic acid encoding a variant E. coli tyrosyl-tRNA synthetase (EcTyr-RS) comprising SEQ ID NO: 1, or an amino acid sequence with at least about 90% sequence identity with the full-length SEQ ID NO:1, wherein the tyrosine (Y) at position 37 is replace with glycine (G) or cysteine (C), the leucine (L) at position 71 is replaced with valine (V), cysteine (C) or isoleucine (I), the aspartic acid (D) at position 182 is replaced with cysteine (C) or serine(S), the phenylalanine (F) at position 183 is replaced with tyrosine (Y) or methionine ((M), the leucine (L) at position 186 is replaced with cysteine (C), arginine (R) or glycine (G), the aspartic acid (D) at position 265 is replaced with arginine (R) and the asparagine (N) at position 126 is conserved. An example of such a nucleic acid is SEQ ID NO: 6 encoding the polyspecific EcTyr-RS-9 (SEQ ID NO: 2). In another embodiment, the kit comprises a variant E. coli tyrosyl-tRNA synthetase comprising an amino acid sequence selected from the group consisting of: SEQ ID NO:2; SEQ ID NO:3; SEQ ID NO: 4 or SEQ ID NO:5.

The kit further comprises one, or more, tyrosyl analogs, wherein the tyrosyl analog is selected from the group consisting of: pBPA, pAEY, pAzAcF, pAzPrAmF, alkene amide, pAcrF, pAlkAcF, pCAcF, pFAcF and propargyl carbamate. The kit can further comprise instructions for producing the protein or peptide of interest.

Some of the Uaas incorporated into the proteins using the polyspecific tyrosyl-RS variants described herein can further comprise bioconjugation handles that allow site-specific labeling of recombinant proteins. These can be used to generate therapeutically relevant protein conjugates (e.g., antibody-drug conjugates) or research reagents (e.g., antibody-fluorophore conjugates). Others are electrophilic amino acids that can create crosslink with natural nucleophilic amino acids, which is useful for many applications, including the ability to make genetically encoded cyclic peptides in cells. Such genetically encoded cyclic peptides can be evolved to develop novel therapeutics.

Embodiments of the disclosure demonstrate features and advantages that will become apparent to one of ordinary skill in the art upon reading the attached Detailed Description.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1. The structures of the UAAs that can be incorporated with the polyspecific TyrRS.

FIG. 2. The polyspecific TyrRS/tRNA pair suppression activity in engineered ATMY E. coli cells for the multiple UAAs that it incorporates, as can be employed in some embodiments. The activity can be measured in embodiments by co-expressing the polyspecific TyrRS/tRNA pair along with a sfGFP reporter harboring a TAG codon at a permissive site. The suppression activity can be evaluated as the full-length sfGFP expression measured using its characteristic fluorescence.

FIG. 3. Yields of sfGFP and EGFP reporters as can be employed in some embodiments. The sfGFP expressions can be carried out as described in the FIG. 2. description. The EGFP expressions can be carried out by co-expressing the polyspecific tyrosyl-tRNA synthetase/tRNA pair in HEK293T cells along with a EGFP reporter harboring a TAG codon. The expressed GFP proteins can then be purified by affinity column chromatography and their yields can be calculated by Bradford assay.

FIG. 4A-N. MS-analysis of the GFP reporter proteins confirming incorporation of all of the UAAs. (A) sfGFP-pBPA (B) EGFP-pBPA (C) sfGFP-LCA (D) EGFP-LCA (E) sfGFP-alpha (F) EGFP-alpha (G) sfGFP-beta (H) EGFP-beta (I) sfGFP-Michael (J) EGFP-Michael (K) sfGFP-alkyne (L) EGFP-alkyne (M) sfGFP-chlor (N) sfGFP-fluoro.

FIG. 5A-B. FIG. 5A shows the active site of the polyspecific TyrRS in the crystal structure (PDB: 1X8X) and the key residues that may be randomized in the active site of E. coli TyrRS to potentially generate polyspecific variants, as may be employed in some embodiments. FIG. 5B shows a table of the key mutations that resulted in polyspecific activities and the sequences of variant poly-tyrosyl-RS proteins (SEQ ID NOS: 2-5).

FIG. 6. Shows the nucleotide sequence of the polyspecific EcTyrRS, poly-TyrRS-9 with mutation sites highlighted in red.

FIG. 7 Shows the amino acid sequence of the wild-type EcTyr-RS (SEQ ID NO: 1).

FIG. 8 Shows the nucleotide sequence of an E. coli Tyr-tRNA (SEQ ID NO:7) suitable for use in the present invention.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The following specific embodiments and examples are to be construed as merely illustrative and not limitative of the remainder of the disclosure in any way whatsoever. Embodiments of the disclosure demonstrate features and advantages that will become apparent to one of ordinary skill in the art upon reading the attached Detailed Description.

The present invention encompasses genetically-engineered mutants/variants of a bacteria-derived tyrosyl-tRNA synthetase (TyrRS) for the efficient incorporation of a variety of UAAs (also referred to herein as ncAAs, non-canonical amino acids) into a protein, polypeptide or peptide of interest, that can be expressed in a bacterial cell or a eukaryotic cell. As described herein, these mutant/variant polyspecific synthetases can incorporate several novel UAAs into proteins or peptides expressed in eukaryotes in a site-specific manner with high fidelity and efficiency. Thus, the present invention, as described herein, enables the expression of eukaryotic proteins or peptides in eukaryotic cells with precisely installed (e.g., incorporated) UAAs at specified positions within the expressed protein or peptide. The ability to incorporate an UAA into virtually any site of any protein or peptide in bacterial and eukaryotic cells offers intriguing opportunities for novel synthetic biology applications.

To produce the variant Ec-tyrosyl-tRNA synthetases described herein, a library was made to genetically-engineer polyspecific tyrosyl-tRNA synthetase variants wherein key active site amino acid residues are randomized (Tyr37, Leu71, Asn126, Asp182, Phe183, Leu186) and can also introduce the point mutation D265 (where in the specific numbering corresponds to the wild-type E. coli tyrosyl-tRNA synthetase (SEQ ID NO:1). (See for example, WO2020/219708 and U.S. Pat. No. 10,717,975, the teachings of which are incorporated herein in their entirety.) The library can be created through site-saturation mutagenesis and can be subjected to a selection scheme in engineered ATM E. coli, yeast, or another suitable host cell, to identify those capable of charging target UAAs with acceptable levels of fidelity and efficiency. Following the selection, individual library members can be characterized for their ability to incorporate the target UAA in response to a nonsense or frameshift codon at various reporter proteins, including, but not limited to chloramphenicol acetyl transferase, GFP, luciferase, etc. Evaluation of library members as described above can identify suitable polyspecific bacteria-derived TyrRS variants that can selectively incorporate multiple different UAAs.

To evaluate the polyspecificity of the engineered tyrosyl-tRNA synthetase, expression of a suitable reporter protein, such as GFP, harboring a nonsense/frameshift codon can be employed in any ATMY E. coli, HEK293T, or other suitable host cells. The suppression activity of the polyspecific TyrRS can be evaluated based on the corresponding reporter protein selectively in the presence of an UAA. The expressed reporter proteins can then be isolated and characterized by mass spectrometry to confirm the incorporation of desired UAAs.

This engineered TyrRS mutant can be used in any eukaryotic cell along with the appropriate cognate tRNA (suppressing a nonsense or frameshift codon, or a codon composed of one or more non-natural nucleobases). Such expression hosts include, but are not limited to, yeast, insect cells, and mammalian cells. Additionally, this TyrRS/tRNA pair can be used for UAA incorporation in engineered ATM E. coli strains, where this bacterial pair has been functionally replaced with a eukaryotic or archaeal counterpart.

The substrate UAAs of the polyspecific TyrRS variants include those with a bioconjugation handle such as alkynes, azides, ketones, cyclopropenes, etc. Site-specific incorporation of these UAAs into proteins will enable their subsequent site-specific labeling through bioorthogonal conjugation reactions. This strategy can be used to generate homogeneous, site-specific conjugates of recombinant proteins, including, but not limited to, antibody-drug conjugates, protein conjugates with various biophysical/biochemical probes such as fluorophores, protein-protein conjugates, protein-nucleic acid conjugates, protein-small molecule conjugates, protein-peptide conjugates, etc.

The substrate UAAs of the polyspecific TyrRS variants include those with electrophilic groups such as α, β-unstaurated carbonyls, halogenated amino acids, etc., which can react with natural amino acid residues in a proximity-dependent manner. Incorporation of such amino acids into proteins can enable covalent capture of a nearby interaction partner. This strategy can also enable the development of proteins (including but not limited to antibodies, protein and peptide ligands for various receptors) that covalently associate with a target for therapeutic or diagnostic applications.

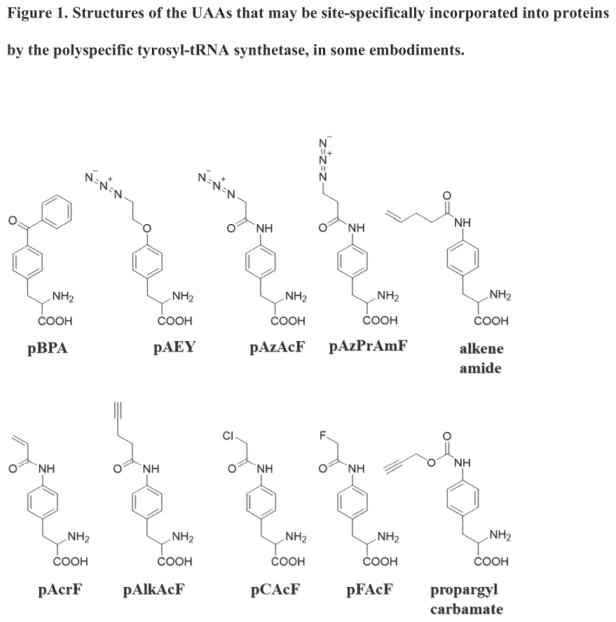

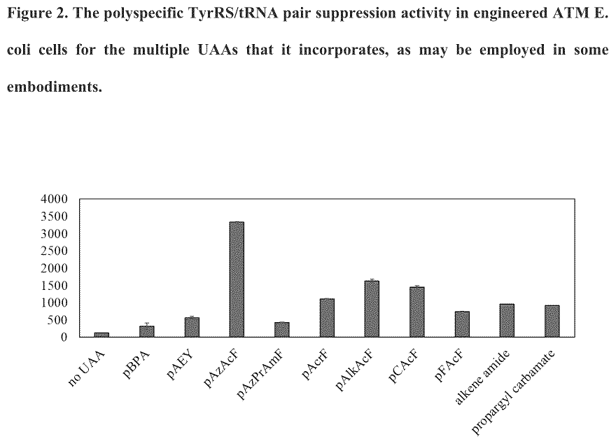

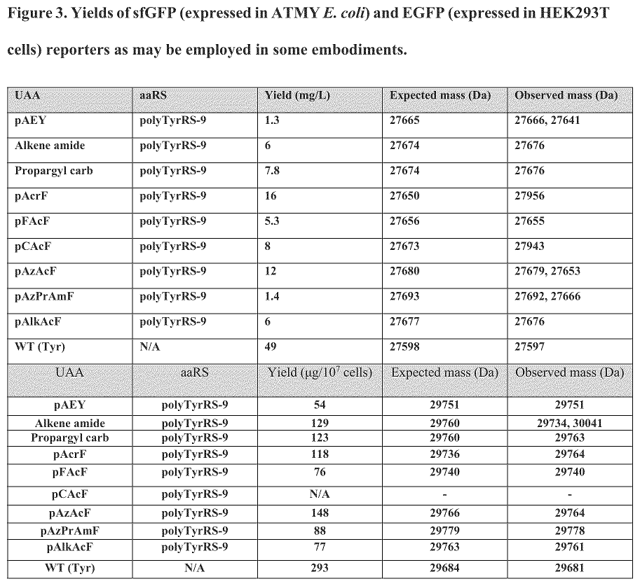

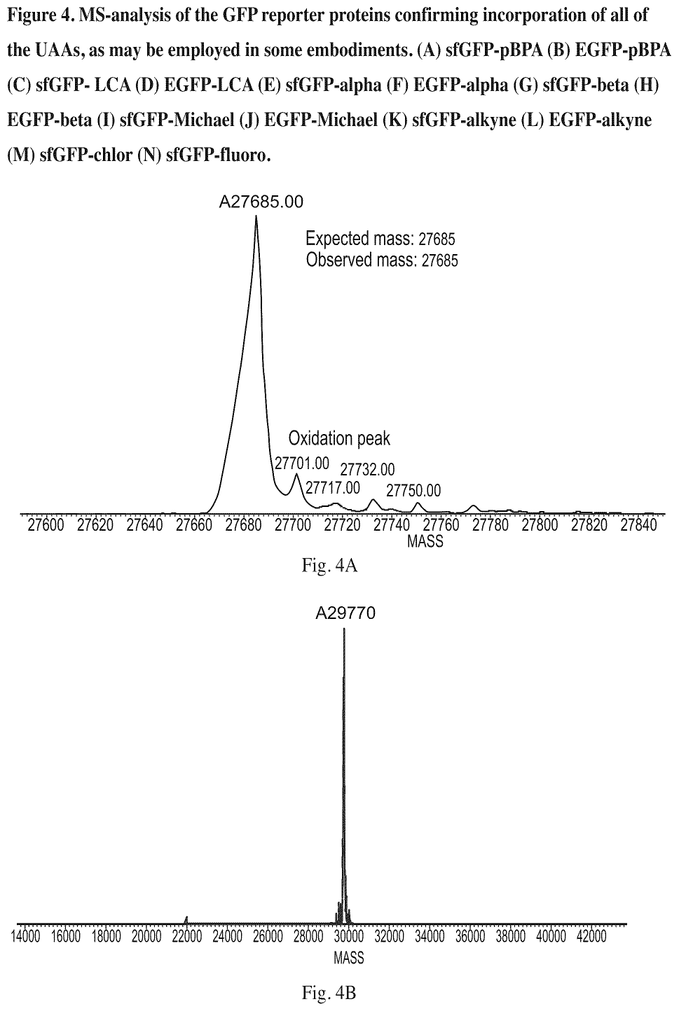

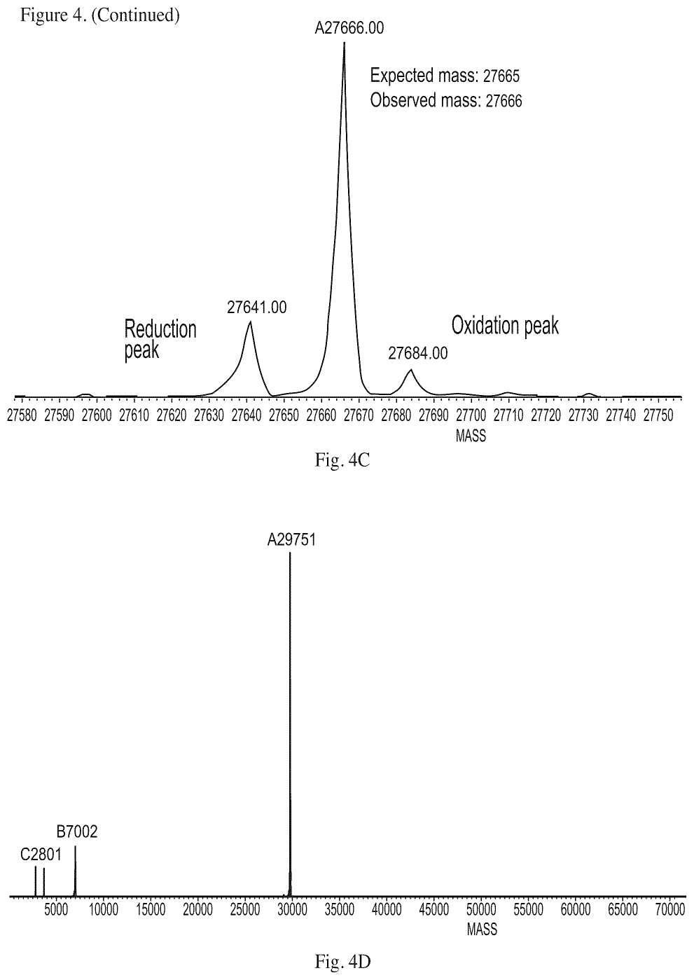

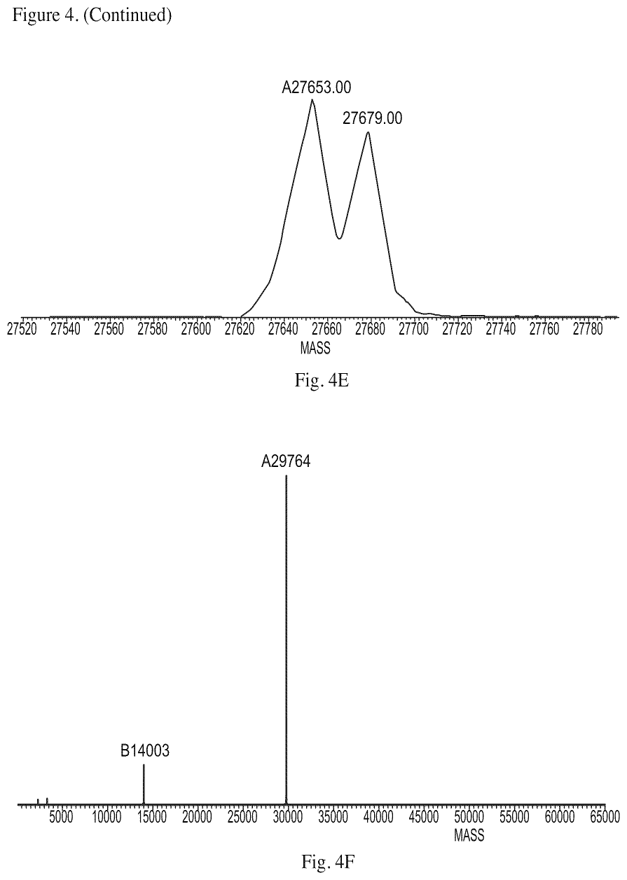

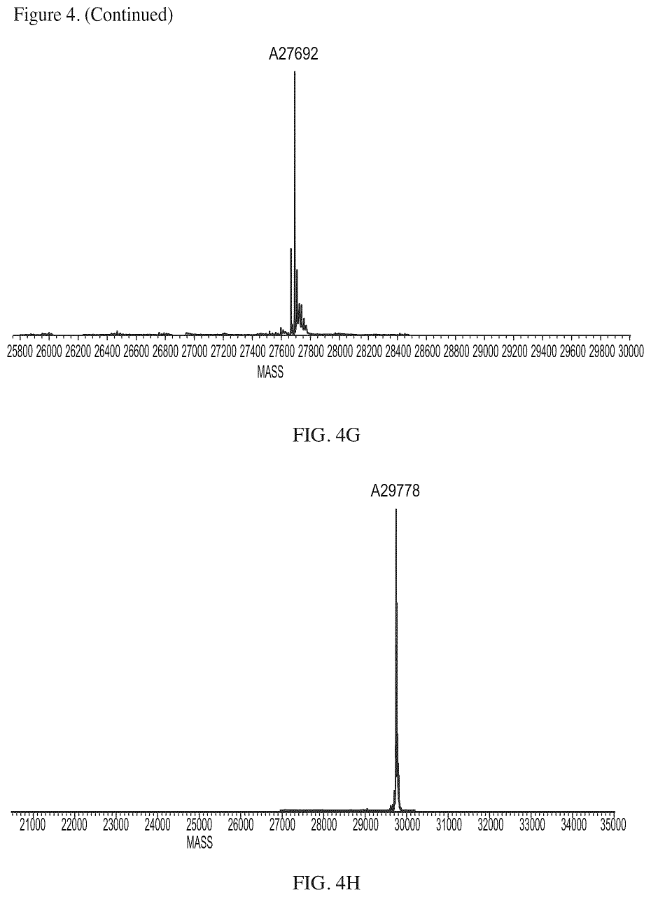

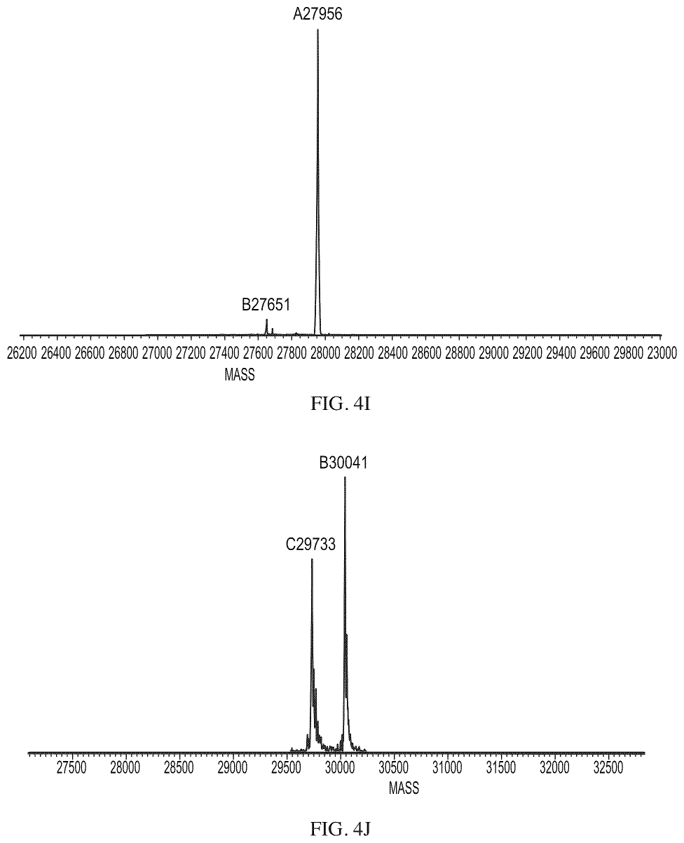

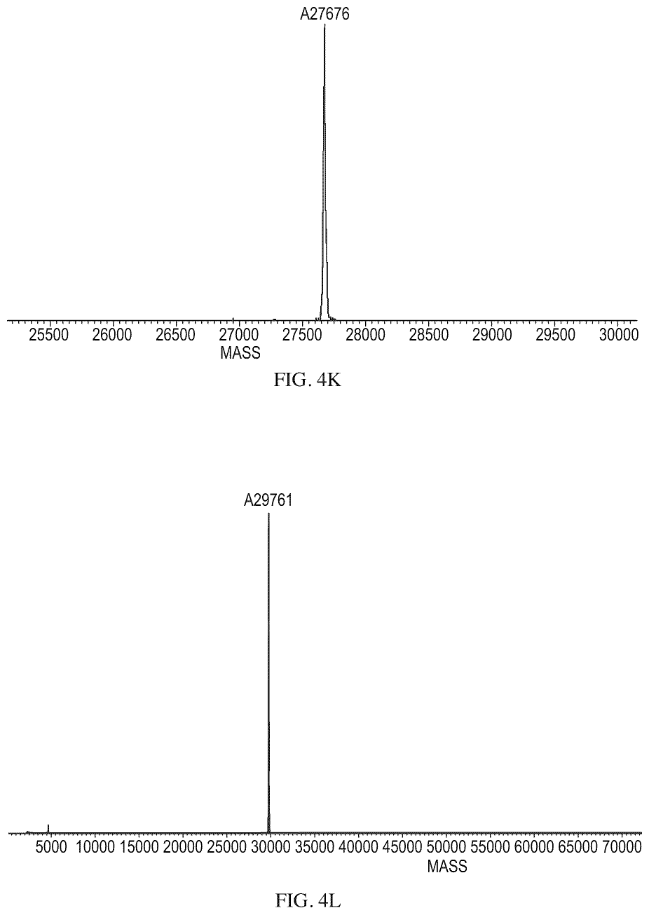

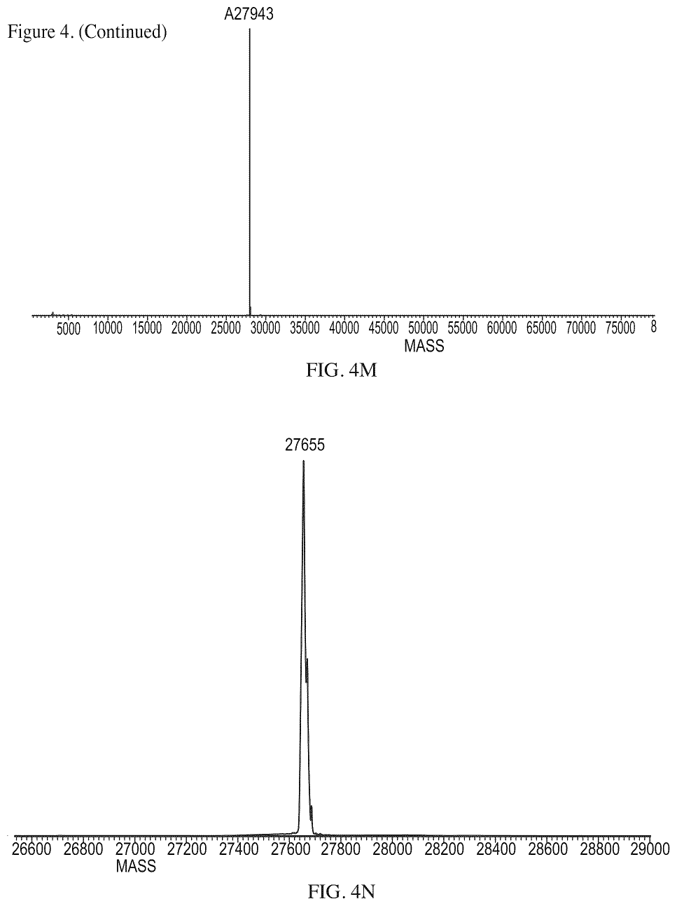

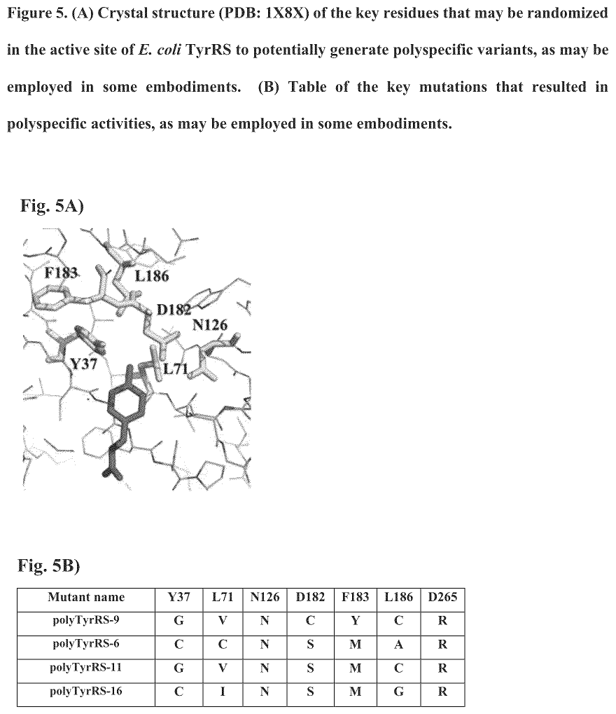

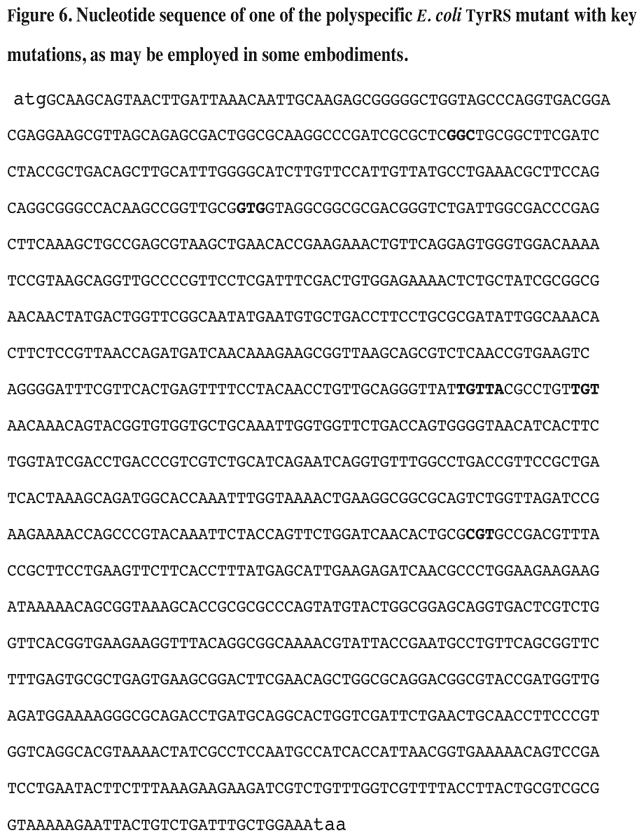

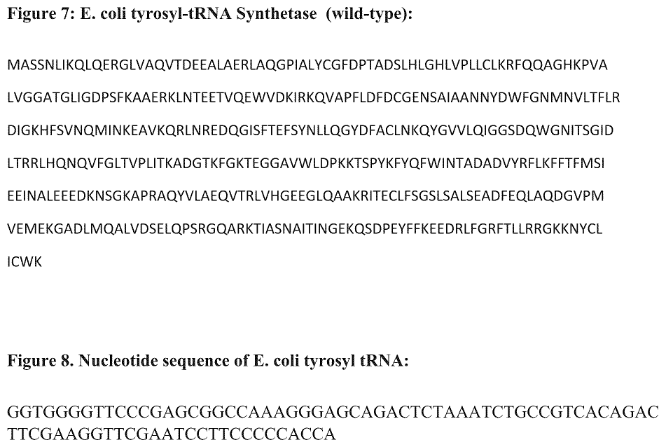

It should be noted that the embodiments described herein should not be limited to these specific bacterial-derived synthetase scaffolds. This disclosure and its embodiments provide a general engineered synthetase active site (FIGS. 5A-B) that can be used for the incorporation of numerous UAAs in both bacterial and mammalian cells (FIGS. 1-4); the cell type, synthetase scaffold, and incorporable UAAs can vary as the technology in this field and in this work advance. Using the techniques described herein, other bacterial tyrosyl-tRNA synthetases can be engineered for UAA incorporation in either bacterial or eukaryotic cells and can include mutations at the following active acid residues that corresponds to the E. coli tyrosyl-tRNA synthetase: Y37; L71; D182; F183; L186; and D265 (FIGS. 5-6).

While this invention has been particularly shown and described with references to preferred embodiments thereof, it will be understood by those skilled in the art that various changes in form and details may be made therein without departing from the scope of the invention encompassed by the appended claims.

1-35. (canceled)

36. A composition comprising a variant E. coli tyrosyl-tRNA synthetase (EcTyr-RS) wherein the variant EcTyr-RS preferentially aminoacacylates an E. coli tyrosyl-RNA (EctRNA^{tyr}) with a tyrosine analog over the naturally-occurring tyrosine amino acid, wherein the variant EcTyr-RS comprises the amino acid sequence of SEQ ID NO:1 or an amino acid sequence with at least about 90% sequence identity with the full-length SEQ ID NO:1, wherein the EcTyr-RS is mutated relative to SEQ ID NO:1 at amino acid residues tyrosine (Y) 37, leucine (L) 71, aspartic acid (D) 182, phenylalanine (F) 183, leucine (L) 186 and aspartic acid (D) 265.

37. The composition of claim 36, wherein the variant E. coli tyrosyl-tRNA synthetase comprises SEQ ID NO: 1, or an amino acid sequence with at least about 90% sequence identity with the full-length SEQ ID NO:1, wherein the tyrosine (Y) at position 37 is replaced with glycine (G) or cysteine (C), the leucine (L) at position 71 is replaced with valine (V), cysteine (C) or isoleucine (I), the aspartic acid (D) at position 182 is replaced with cysteine (C) or serine(S), the phenylalanine (F) at position 183 is replaced with tyrosine (Y) or methionine ((M), the leucine (L) at position 186 is replaced with cysteine (C), arginine (R) or glycine (G), the aspartic acid (D) at position 265 is replaced with arginine (R) and the asparagine (N) at position 126 is conserved.

38. The composition of claim 37, wherein the variant E. coli tyrosyl-tRNA synthetase comprises an amino acid sequence selected from the group consisting of: SEQ ID NO:2; SEQ ID NO:3; SEQ ID NO: 4 or SEQ ID NO:5.

39. A polynucleotide encoding a variant E. coli tyrosyl-tRNA synthetase of claim 38, wherein the nucleotide sequence comprises SEQ ID NO:6.

40. The composition of claim 36, wherein the tyrosine analogs are selected from the group consisting of: pBPA, pAEY, pAzAcF, pAzPrAmF, alkene amide, pAcrF, pAlkAcF, pCAcF, pFAcF and propargyl carbamate.

41. A cell comprising a variant E. coli tyrosyl-tRNA synthetase (EcTyr-RS), wherein the variant EcTyr-RS preferentially aminoacylates an E. coli tyrosyl-tRNA with a tyrosyl analog, and an orthogonal E. coli-tyrosyl tRNA (Ec-tRNA^{Tyr}) as a pair, wherein the variant EcTyr-RS comprises the amino acid sequence of SEQ ID NO: 1 or an amino acid sequence with at least about 90% sequence identity with the full-length SEQ ID NO:1, wherein the EcTyr-RS is mutated relative to SEQ ID NO: 1 at amino acid residues tyrosine (Y) 37, leucine (L) 71, aspartic acid (D) 182, phenylalanine (F) 183, leucine (L) 186 and aspartic acid (D) 265.

42. The cell of claim 41, wherein the variant EcTyr-RS comprises the amino acid sequence SEQ ID NO:1, or an amino acid sequence with at least about 90% sequence identity with the full-length SEQ ID NO:1, wherein the tyrosine (Y) at position 37 is replace with glycine (G) or cysteine (C), the leucine (L) at position 71 is replaced with valine (V), cysteine (C) or isoleucine (I), the aspartic acid (D) at position 182 is replaced with cysteine (C) or serine(S), the phenylalanine (F) at position 183 is replaced with tyrosine (Y) or methionine ((M), the leucine (L) at position 186 is replaced with cysteine (C), arginine (R) or glycine (G), the aspartic acid (D) at position 265 is replaced with arginine (R) and the asparagine (N) at position 126 is conserved.

43. The cell of claim 42, wherein the variant E. coli tyrosyl-tRNA synthetase comprises an amino acid sequence selected from the group consisting of: SEQ ID NO: 2; SEQ ID NO:3; SEQ ID NO: 4 or SEQ ID NO:5.

44. The cell of claim 43, wherein the cell comprises a polynucleotide encoding the amino acid sequence of the variant E. coli tyrosyl-tRNA synthetase.

45. The cell of claim 44, wherein the polynucleotide comprises SEQ ID NO:6, or a nucleotide sequence with at least about 90% sequence identity of SEQ ID NO: 6.

46. The cell of claim 41, wherein the cell is an E. coli cell, a eukaryotic cell or a mammalian cell.

47. The cell of claim 41, wherein the tyrosine analog is selected from the group consisting of: pBPA, pAEY, pAzAcF, pAzPrAmF, alkene amide, pAcrF, pAlkAcF, pCAcF, pFAcF and propargyl carbamate.

48. A method of producing a protein or peptide of interest in an E. coli, eukaryotic or mammalian cell with one, or more, tyrosyl analogs at specified amino acid residue positions in the protein or peptide, the method comprising,
a. culturing the cell in a culture medium under conditions suitable for growth, wherein the cell comprises a nucleic acid encoding a protein or peptide of interest with one, or more, selector codons incorporated at the one, or more specified positions in the protein or peptide, wherein the cell further comprises a nucleic acid encoding an Ec-tRNA^{Tyr} that recognizes the selector codon, and
b. contacting the cell culture medium with one, or more, tyrosyl analogs under conditions suitable for incorporation of the one, or more, tyrosyl analogs into the protein in response to the selector codon,
thereby producing the protein or peptide of interest with one, or more tyrosyl analogs at specified positions in the protein or peptide.

49. The method of claim 48, wherein the tyrosyl analog is selected from the group consisting of: pBPA, pAEY, pAzAcF, pAzPrAmF, alkene amide, pAcrF, pAlkAcF, pCAcF, pFAcF and propargyl carbamate.

50. The method of claim 48, wherein the cell further comprises a second ERNA/RS pair that is orthogonal to the cell, wherein the second pair does not cross-react with the EcTyr-RS/tRNA pair and that recognizes an amber selector codon in the protein, wherein the protein produced contains one, or more tyrosyl analogs and one, or more, distinct unnatural amino acid other than a tyrosyl analog.

51. A method of site-specifically incorporating one, or more, tyrosyl analogs into a protein or peptide of interest in an E. coli, eukaryotic or mammalian cell, the method comprising,
a. culturing the cell in a culture medium under conditions suitable for growth, wherein the cell comprises a nucleic acid encoding a protein or peptide of interest with one, or more, amber selector codons incorporated at one, or more at specific sites in the protein or peptide, and wherein the cell further comprises a nucleic acid encoding a variant E. coli tyrosyl-tRNA synthetase (EcTyr-RS), wherein the EcTry-RS preferentially aminoacylates an E. coli tyrosyl tRNA (Ec-tRNA^{Try}) that recognizes the amber selector codon, wherein the variant EcTyr-RS comprises the amino acid sequence selected from the group consisting of: SEQ ID NO:2, SEQ ID NO: 3, SEQ ID NO:4 or SEQ ID NO:5; and
b. contacting the cell culture medium with one, or more, tyrosyl analogs under conditions suitable for incorporation of the one, or more, tyrosyl analogs into the protein or peptide at the sites of the selector codon(s),
thereby producing the protein or peptide of interest with one, or more site-specifically incorporated tyrosyl analogs.

52. The method of claim 51, wherein the tyrosyl analog is selected from the group consisting of: pBPA, pAEY, pAzAcF, pAzPrAmF, alkene amide, pAcrF, pAlkAcF, pCAcF, pFAcF and propargyl carbamate.

53. The method of claim 51, wherein the cell further comprises a second tRNA/RS pair that is orthogonal to the cell, wherein the second pair does not cross-react with the EcTyr-RS/tRNA pair and that recognizes an amber selector codon in the protein, wherein the protein or peptide of interest produced contains one, or more tyrosyl analogs. and one, or more, distinct unnatural amino acid residues other than a tyrosyl residue.

54. A kit for producing a protein or peptide of interest in a cell, wherein the protein or peptide comprises one, or more tyrosyl analogs, the kit comprising:
a. a container containing a polynucleotide sequence encoding an Ec-tRNA^{Tyr} that recognizes an amber selector codon; and
b. a container containing a polynucleotide encoding a variant E. coli tyrosyl tRNA synthetase that preferentially aminoacylates the Ec-tRNA^{Tyr} with a tryrosyl analog, wherein the EcTry-RS comprises an amino acid sequence selected from the group consisting of: SEQ ID NO:2, SEQ ID NO:3, SEQ ID NO: 4 or SEQ ID NO:5.

55. The kit of claim 54, wherein the kit further comprises one, or more, tyrosyl analogs, wherein the tyrosyl analog is selected from the group consisting of: pBPA, pAEY, pAzAcF, pAzPrAmF, alkene amide, pAcrF, pAlkAcF, pCAcF, pFAcF and propargyl carbamate.


SEQUENCE LISTING (7 sequences)

SEQ ID NO: 1 - protein, 424 aa, Escherichia coli
MASSNLIKQLQERGLVAQVTDEEALAERLAQGPIALYCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYDFACLNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 2 - protein, 424 aa, Escherichia coli
MASSNLIKQLQERGLVAQVTDEEALAERLAQGPIALGCGFDPTADSLHLGHLVPLLCLKRFQQAGHKPVAVVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYCYACCNKQYGVVLQIGGSDQWGNITSGIDLTRRLHQNQVFGLTVPLITKADGTKFGKTEGGAVWLDPKKTSPYKFYQFWINTARADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 3 - protein, 424 aa, Escherichia coli
MASSNLIKQLQERGLVAQVTDEEALAERLAQGPIALCCGFDPTADSLHLGHLVPLLCLKRFQQAGHKPVACVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYSMACANKQYGVVLQIGGSDQWGNITSGIDLTRRLHQNQVFGLTVPLITKADGTKFGKTEGGAVWLDPKKTSPYKFYQFWINTARADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 4 - protein, 424 aa, Escherichia coli
MASSNLIKQLQERGLVAQVTDEEALAERLAQGPIALGCGFDPTADSLHLGHLVPLLCLKRFQQAGHKPVAVVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYSMACCNKQYGVVLQIGGSDQWGNITSGIDLTRRLHQNQVFGLTVPLITKADGTKFGKTEGGAVWLDPKKTSPYKFYQFWINTARADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 5 - protein, 424 aa, Escherichia coli
MASSNLIKQLQERGLVAQVTDEEALAERLAQGPIALCCGFDPTADSLHLGHLVPLLCLKRFQQAGHKPVAIVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYSMACGNKQYGVVLQIGGSDQWGNITSGIDLTRRLHQNQVFGLTVPLITKADGTKFGKTEGGAVWLDPKKTSPYKFYQFWINTARADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 6 - DNA, 1275 bp, Escherichia coli
  source 1..1275: mol_type = genomic DNA
atggcaagcagtaacttgattaaacaattgcaagagcgggggctggtagcccaggtgacggacgaggaagcgttagcagagcgactggcgcaaggcccgatcgcgctcggctgcggcttcgatcctaccgctgacagcttgcatttggggcatcttgttccattgttatgcctgaaacgcttccagcaggcgggccacaagccggttgcggtggtaggcggcgcgacgggtctgattggcgacccgagcttcaaagctgccgagcgtaagctgaacaccgaagaaactgttcaggagtgggtggacaaaatccgtaagcaggttgccccgttcctcgatttcgactgtggagaaaactctgctatcgcggcgaacaactatgactggttcggcaatatgaatgtgctgaccttcctgcgcgatattggcaaacacttctccgttaaccagatgatcaacaaagaagcggttaagcagcgtctcaaccgtgaagatcaggggatttcgttcactgagttttcctacaacctgttgcagggttattgttacgcctgttgtaacaaacagtacggtgtggtgctgcaaattggtggttctgaccagtggggtaacatcacttctggtatcgacctgacccgtcgtctgcatcagaatcaggtgtttggcctgaccgttccgctgatcactaaagcagatggcaccaaatttggtaaaactgaaggcggcgcagtctggttagatccgaagaaaaccagcccgtacaaattctaccagttctggatcaacactgcgcgtgccgacgtttaccgcttcctgaagttcttcacctttatgagcattgaagagatcaacgccctggaagaagaagataaaaacagcggtaaagcaccgcgcgcccagtatgtactggcggagcaggtgactcgtctggttcacggtgaagaaggtttacaggcggcaaaacgtattaccgaatgcctgttcagcggttctttgagtgcgctgagtgaagcggacttcgaacagctggcgcaggacggcgtaccgatggttgagatggaaaagggcgcagacctgatgcaggcactggtcgattctgaactgcaaccttcccgtggtcaggcacgtaaaactatcgcctccaatgccatcaccattaacggtgaaaaacagtccgatcctgaatacttctttaaagaagaagatcgtctgtttggtcgttttaccttactgcgtcgcggtaaaaagaattactgtctgatttgctggaaataa

SEQ ID NO: 7 - DNA, 82 bp, Escherichia coli
  source 1..82: mol_type = other DNA
ggtggggttcccgagcggccaaagggagcagactctaaatctgccgtcacagacttcgaaggttcgaatccttcccccacca